(19) United States
(12) Patent Application Publication
(10) Pub. No.: US 2021/0262130 A1
(43) Pub. Date: Aug. 26, 2021

(54) NON-SHEDDING HYBRID NONWOVENS AND METHOD OF PRODUCING SAME

(71) Applicant: Groz-Beckert KG, Albstadt (DE)

(72) Inventors: Behnam Pourdeyhimi, Cary, NC (US); Hooman Amid, Raleigh, NC (US); Benoit Maze, Raleigh, NC (US); Michael C. Flickinger, Port Ludlow, WA (US)

(21) Appl. No.: 17/261,719

(22) PCT Filed: Jul. 17, 2019
(86) PCT No.: PCT/IB2019/000769

§ 371 Date: Jan. 20, 2021

Related U.S. Application Data
(60) Provisional application No. 62/701,333, filed on Jul. 20, 2018.

(51) Int. Cl. D04H 1/407; D04H 1/56; D04H 3/16; B01D 39/16

(52) U.S. Cl. CPC D04H 1/407; D04H 1/56; D04H 3/16; B01D 39/1623; B01D 2239/0407; B01D 2239/10; B01D 2239/0618; B01D 2239/1233; B01D 2239/0668; B01D 2239/0622; B01D 2239/1241

(57) ABSTRACT

A non-shedding hybrid nonwoven web comprised of 99% by mass of functional material(s) co-mingled with the filaments. High level of particle retention within the fabric is provided without the aid of adhesives, binders, adhesive polymers, or post processes. This composite web has a multi-layered structure with a uniform distribution of sorptive particles and desired opposed color contrast on each side. The process includes providing two converging streams of blown polymeric filaments, passive feed of functional material(s) in between the filament streams, and collecting the hybrid webs.

### TECHNICAL FIELD

The subject matter disclosed herein relates generally to hybrid nonwoven fabrics used in applications wherein high mass concentrations of non-shedding functional materials are desirable such as adsorptive filters and media, liquid purification media, absorptive wipes, diapers, odor-removal media, and the like. More particularly, the present subject matter relates to hybrid nonwoven fabrics and methods for manufacturing thereof wherein the functional component(s) is wrapped around by the fiber component(s) and physically ensnared without the use of adhesive(s) or adhesive polymer(s), therefore its surface functionality is fully preserved.

### BACKGROUND

Hybrid nonwovens are fibrous structures that contain functional materials. The fibrous component may consist of one or more different fibrous constituents, i.e. fibers that are made from different polymeric materials, cross sectional shapes, bulk densities, and/or fiber diameters. At the same time, the functional component may be from one or more different constituents, with distinct physical/chemical properties. Exemplary materials include, but are not limited to, activated carbon particles, superabsorbent particles, crimped/bulky staple fibers, wood pulp fibers, metal organic frameworks (MOFs), and so forth.

Depending on the type of functional materials, applications of hybrid nonwovens encompass a wide variety of market segments. For instance, activated carbon loaded substrates provide high specific surface areas, which are useful for adsorbing unwanted toxic molecules and odors, adsorption of precious metal particles, storage of hydrogen gas molecules, and different catalytic activities and applications. Hybrid nonwoven substrates with wood pulp staple fibers and/or superabsorbent polymers have many applications in diapers and wiping products. In addition, hybrid nonwovens that are loaded with crimped and bulky staple fibers provide great thermal/acoustic insulation properties. These are just a few examples of many commercial and industrial applications of hybrid (or composite) nonwovens.

Conventional methods to manufacture hybrid nonwovens employ traditional nonwoven production and finishing techniques. The most popular technology for previously-formed substrates is the dip and squeeze method, i.e. dipping the nonwovens web/mat in a suspension of functional and binder materials, squeezing the excess binder material (typically acrylic based), and curing the final composite structure. Coating is another popular method, where the pre-formed substrate is coated with the functional material(s). Although the sequences may vary, the general procedure is to apply or coat the substrate with the functional material(s). The binder material may be applied before, after, with, or at all times as the functional material is being introduced to the substrate. Alternatively, for not-pre-formed substrates, the functional material is mixed with the fibers in the wetlay or airlay process, to form the composite mat. To ensure that the particles are sufficiently attached to the fibers, bicomponent core-sheath fibers or two types of fibers may be used, wherein the sheath or the second fiber type has a lower melting temperature than the core or the first fiber type, and wherein the hybrid web is later heated to fuse the particles to the soften said sheath (or second type) fibers. In all of the above cases, to ensure that the hybrid web does not release the particles during handling and/or application (particle shedding), the final composite webs are laminated with one layer (or layers) of “backing” textile material, or sandwiched between two layers of textile materials (see FIG. 1).

A more recent technology to produce hybrid nonwovens is the meltblown (or spunblown) coform process. This concept was firstly introduced by Exxon in 1954, wherein an air-jet of staple fibers was introduced to the polymeric filaments, as they were blown to form the nonwoven web. Ever since, the coform configuration and process parameters were modified and/or improved by 3M and Kimberly-Clark. One important improvement was to introduce the air-jet stream of fibers/particles in between two converging meltblown filament streams. This two-beam configuration ensured better co-mingling between the filaments and the functional component(s) (see FIG. 2). However, this final web still requires more processes to prevent the unwanted release of the particles.

There are three important configurational parameters that influence the quality of two-beam coform webs, i) the direction of particle/fiber feed relative to the filament streams, ii) the symmetry of the configuration, and iii) the velocity of the particles relative to the filament streams. From the above parameters, prior art has only discussed the influence of relative velocity. The velocity of the particles relative to the filament streams, determine the three-dimensional distribution uniformity of the particles within a coform structure. Particles with higher relative velocities create a gradient distribution through the web thickness, and vice versa.

Regardless of the particles relative velocity, an active means, such as air-flow, fluidized beds, and so forth, provides the source for such particle acceleration. The common belief is that the particles can overcome the turbulent attenuating air-flow, and entrain the filament stream(s), only if they are actively blown towards the stream(s). The coform webs in which the particles are passively dropped-in (or shaken onto) the meltblown web, are susceptible to shedding, unless adhesive meltblown filaments and/or carrier layers are used.

There are some limitations regarding the reliability of the manufacturing process, and stability of the hybrid nonwoven structures. Reliable and stable process/structure refers to maximized particle loading and minimized particle shedding, with the least number of involved processes, while the functionality of the web/particle is not compromised (i.e. covering the material's surface area and/or diminishing (or adversely altering) their mechanochemical properties. Conventional methods to produce hybrid nonwovens (dip-squeeze, coating, air/wet lay bicomponent fibers, lamination, and sandwiching) involve the use of binder materials and/or heat bonding; both of which cover (or mask) the active specific surface area of the functional materials. Not only these are multi-step processes, but also the typical mass concentration of such “glued” particles is less than 80%. Spunblown coform nonwovens can be produced without the application of binder materials; however, they still need heat-bonded backing/carrier layers or adhesive polymeric fibers to support the particles. For instance, a coform web with 80% mass concentration of carbon particles (web total basis weight: 375 g/m²) may only retain 87% of its particles after a shake-out test (particle shedding ˜13%).

Therefore, it is commonly believed that the particles cannot be incorporated passively, and regardless of the incorporation method, lamination, adhesive/binder material, heat-bonding, or other post processes are required to securely maintain the particles within the coform structure. Considering the prior art about meltblown coform technology, there is limited (or no) knowledge about the following information:

1) Potential influence of the presence of particle/fiber feeding unit in the meltblown vicinity (i.e. disturbance for the fiber/web formation process);

2) Influence of the direction of particle/fiber feed relative to meltblown filament streams;

3) Effect of non-axisymmetric filament streams on particle entrainment and co-mingling;

4) Limits for particle loading, particle shedding, and hybrid web basis weight for passive particle incorporation;

5) Quantitative analysis of the surface area coverage (i.e. the extent at which the filaments and/or coform process cover the particles' surface area).

Meltblown coform nonwovens have widespread applications, and there is a big commercial opportunity and value for the manufacturing know-how of non-shedding particle-comingled webs, without the use of binder/adhesive materials. The subject matter of this disclosure addresses the above missing information and provides detailed description for the manufacture of high-loading non-shedding coform webs.

### SUMMARY

In broad summary, herein is disclosed a non-shedding coform structure with about 99% mass concentration of activated carbon particles, wherein neither adhesive/binder material, nor lamination/post-processes have been used. The particles are passively introduced to the filament streams, and the fiber/web formation process is not disturbed. As opposed to the common belief, the dual-beam coform configuration of this disclosure accommodates the passive particle entrainment and co-mingling, at high loadings and minimal shedding. We believe that this is due to the non-axisymmetric configuration of our coform process, and the optimized location for particle introduction. The non-axisymmetric configuration results in unbalanced (not equal) meltblown vortices. The unbalanced vortices provide the opportunity to introduce passively-fed particles without substantial particle loss during the process. One meltblown stream captures the particles, and the secondary stream helps further comingling them with both of the streams. Compared to the state-of-the-art coform nonwovens, the structure is multi-layered, wherein particles are enwrapped with individual filaments (mechanically trapped). This close entrapment holds the particles in place, preventing particle shed-out from the web and/or particle migration within the web. The entrapment is mechanical, not heat-bonding/infusion, therefore the surface area of the activated carbon is not covered.

### BRIEF DESCRIPTION OF THE DRAWINGS

FIG. 1.1 is the schematic of such structures from prior art, FIG. 1.2 is the cross sectional SEM micrograph of a sandwiched structure, and FIG. 1.3 is the top view of an airlaid structure from prior art;

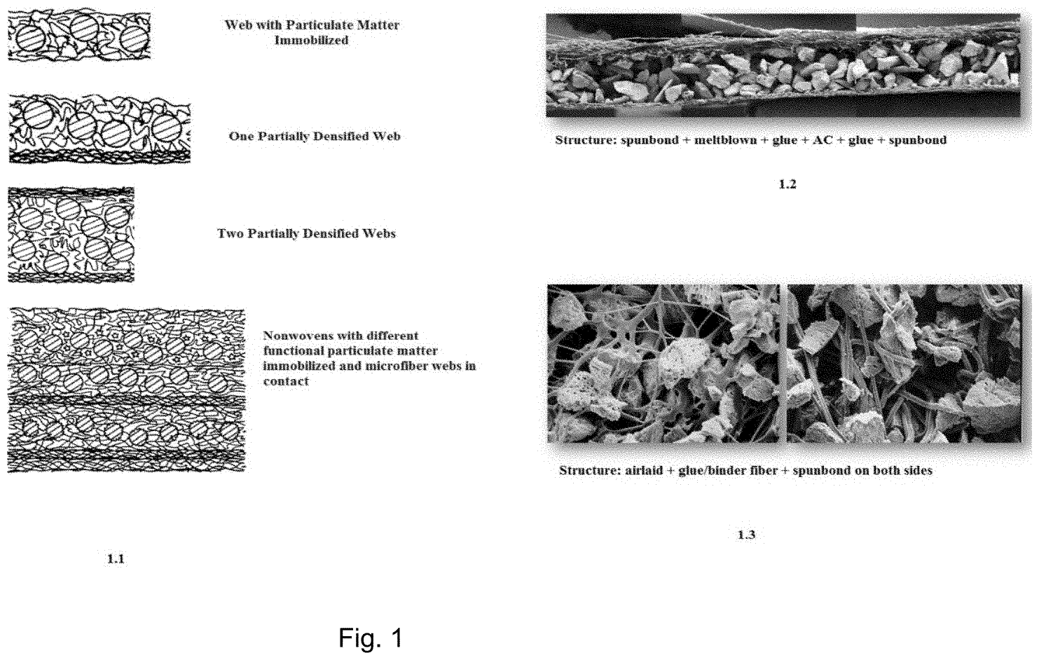

FIG. 2 is the schematic drawing of reported dual-beam coform configurations;

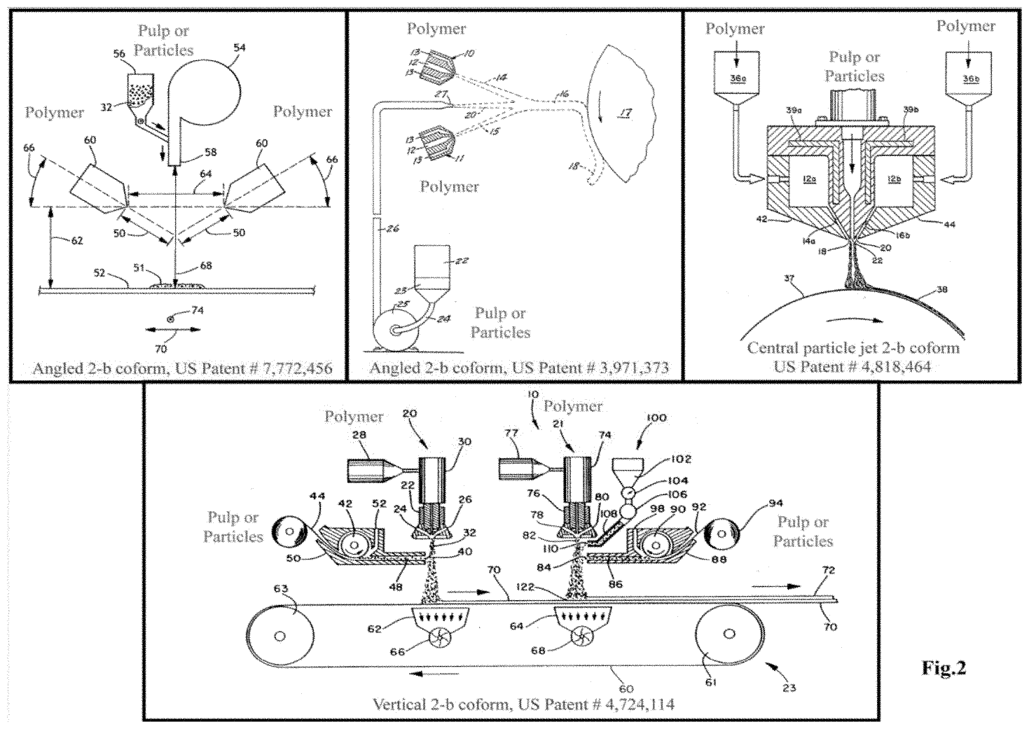

FIG. 3 illustrates unsuccessful passive particle incorporation in axisymmetric coform configurations;

FIG. 4 illustrates unbalanced velocity contours in the converging volume of non-axisymmetric filament streams;

FIG. 5 illustrates entrainment of the AC particles in the streams of converging filaments;

FIG. 6 illustrates an example of a multi-layer structure of the disclosed hybrid web;

FIG. 7 illustrates three coform configurations for high-loading hybrid webs;

FIG. 8 illustrates particle loading and production waste for high-loading nonwovens;

FIG. 9 illustrates shedding parameters of the hybrid webs and CBM;

FIG. 10 illustrates locations for particle discharge in the converging volume

### DETAILED DESCRIPTION

A hybrid nonwoven fabric is manufactured via meltblown coform process, wherein activated carbon particles are co-mingled with thermoplastic filaments. Individual, or a few number of, particles are enwrapped by a plurality of non-adhesive filaments, which results in virtually zero particle release (shedding) and/or particle migration within the entangled web structure. The method includes introduction and incorporation of particles in the nonwoven web, as the filaments are meltblown. Although the particles are dropped into the web based on their weight (gravitational force), they successfully entrain the high velocity filament streams and comingle with the filaments, with essentially no processing waste.

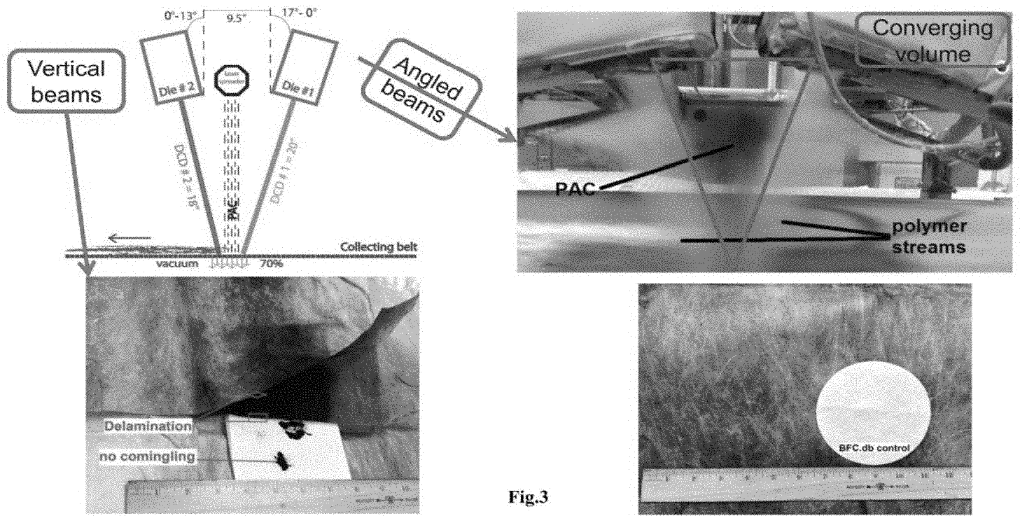

The subject matter disclosed herein relates to improving particle retention within meltblown coform nonwovens at high particle mass concentrations (>98%). In addition, using the disclosed coform configuration, hybrid coform webs are manufactured in one step, and no further post-processes are required to fixate the particles within the web. Typical dual-beam coform process is done in axisymmetric configurations, and it is commonly believed that passive particle introduction in such geometries would lead to excessive particle waste/drop-out, and/or supporting backing layers or adhesive filaments are required (See FIG. 3).

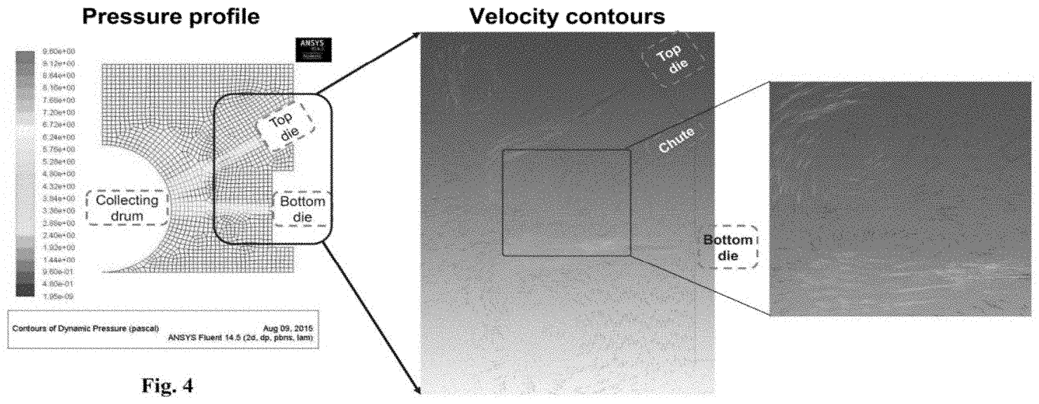

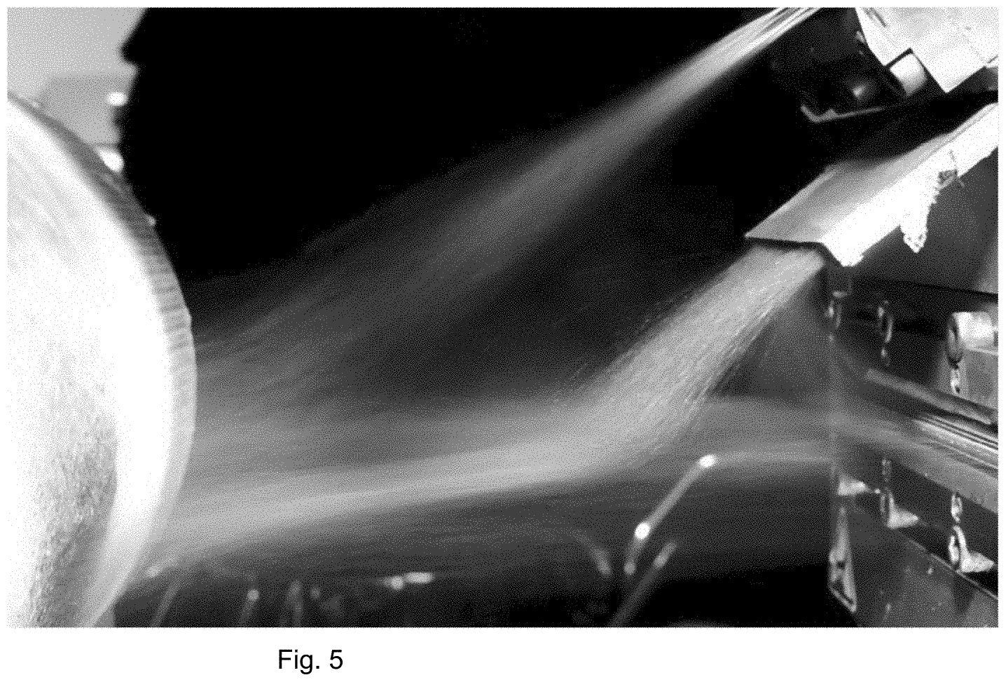

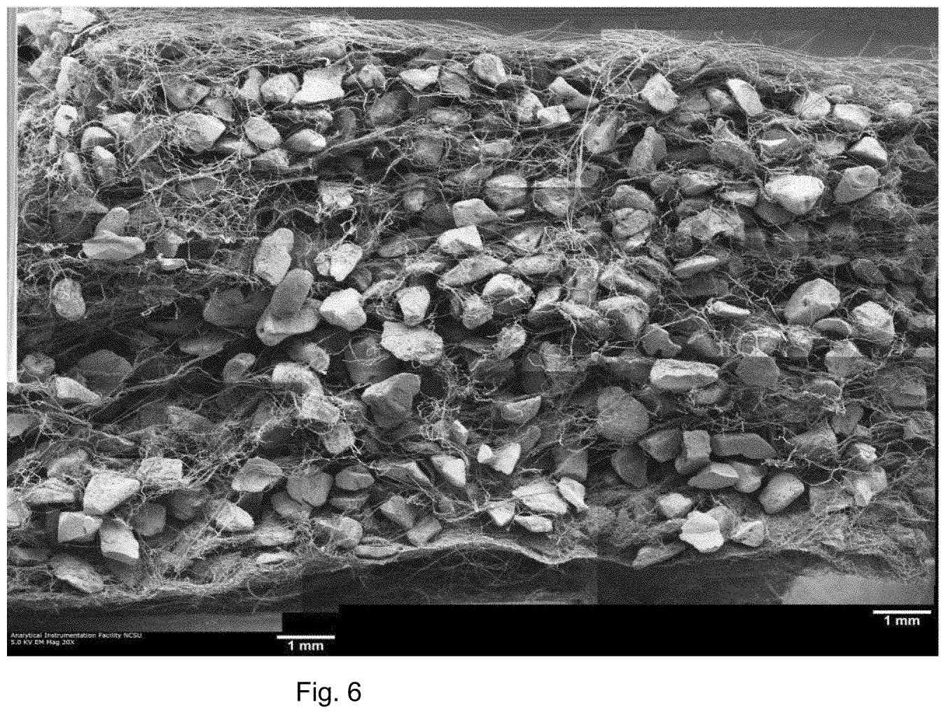

The inventors have discovered that in the disclosed non-axisymmetric configuration, the meltblown vortices are unbalanced. This means the opportunity for uninterrupted passive introduction of particles before the convergence of filament streams, and before either of the vortices are created (see FIG. 4). The particles fall into the bottom filament stream, and while they are moving toward the web collection surface, in this case a rotating drum with internal suction for web retention on the surface, some of them start comingling with some of the filaments. As this partially-coformed web gets closer to the collecting surface, the top filament stream joins the structure, intermingles with the bottom filaments, and comingles with the particles [see FIG. 5]. In other words, the bottom layer acts as an initial filter that captures the loose particles, and moves them forward to further secure them by the aid of top filament stream. This forms a multi-layered structure wherein particles are secured, mechanically entrapped, by the filaments in a uniform manner in the z-direction [see FIG. 6]. Hence, there is no need for adhesive polymers, supporting or backing structures, and/or post-processes. This phenomenon and such multi-layered structures were not expected, and result in a single-step high-throughput coform process.

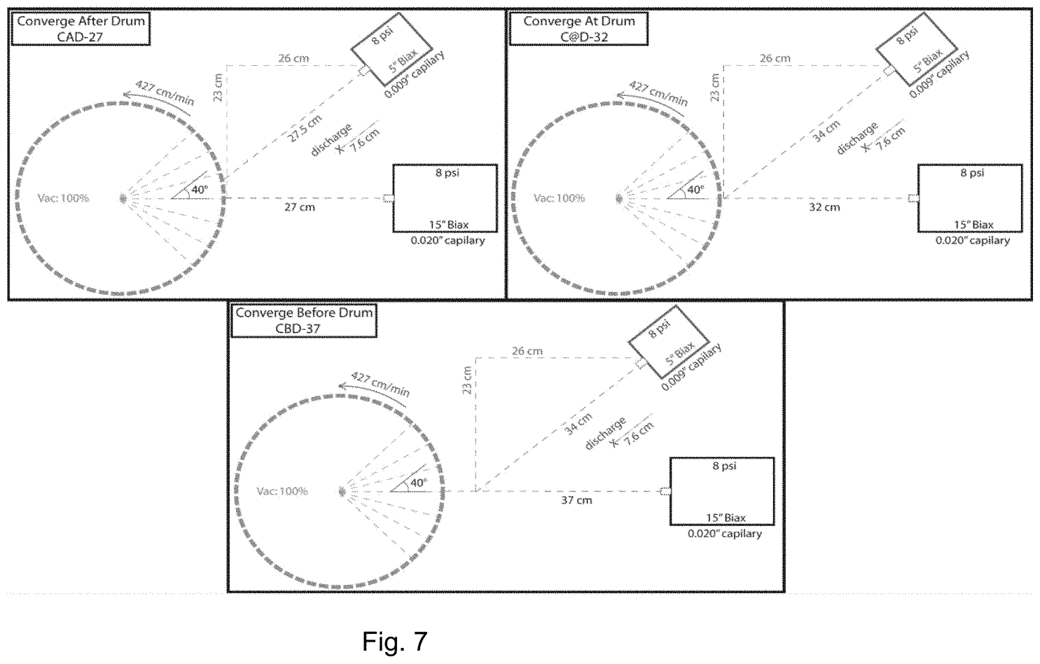

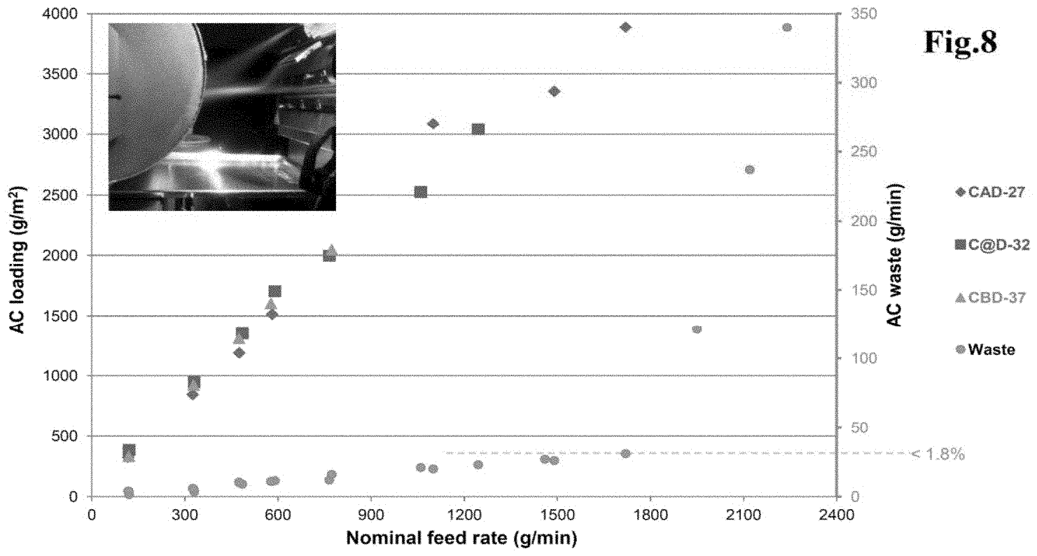

The location at which the filament streams converge had a significant influence on the degree of particle comingling and fiber intermingling. This was controlled by die-to-collector distance (DCD), while the relative angle of the meltblown beams and the particle discharge chute was held constant (see FIG. 7). In one embodiment, DCD=27 cm-CAD-27, filaments did not converge before wind-up. In this configuration, the bottom filaments comingled with the particles and the filaments form the top beam laid on top of the comingled layer. The thickness of the hybrid web was about 60% higher than that of the webs that converged at/before wind-up. In addition, the top filaments had minimal appearance on the bottom side of the hybrid web. In another embodiment, DCD=32 cm-C@D-32, filaments converged right at the wind-up on the collection drum, which showed lower thickness and a better intermingling and co-mingling effect. In the preferred embodiment, DCD=37 cm-CBD-37, filaments converged before arriving to the drum, where they enwrapped individual (or groups) of activated carbon particles, intermingled with each other, and created a stable multi-layered hybrid structure. This structure was a surprise to the inventors, as it was expected that the heavy mass of activated carbon particles would break the filament stream and fall through. However, the unbalanced vortices distributed the particles within the structure and collected them with less than 2% waste, where 1 gram of nonwoven web contained 99 grams of activated carbon particles (see FIG. 8). Particle retention in the final webs was more than 99%.

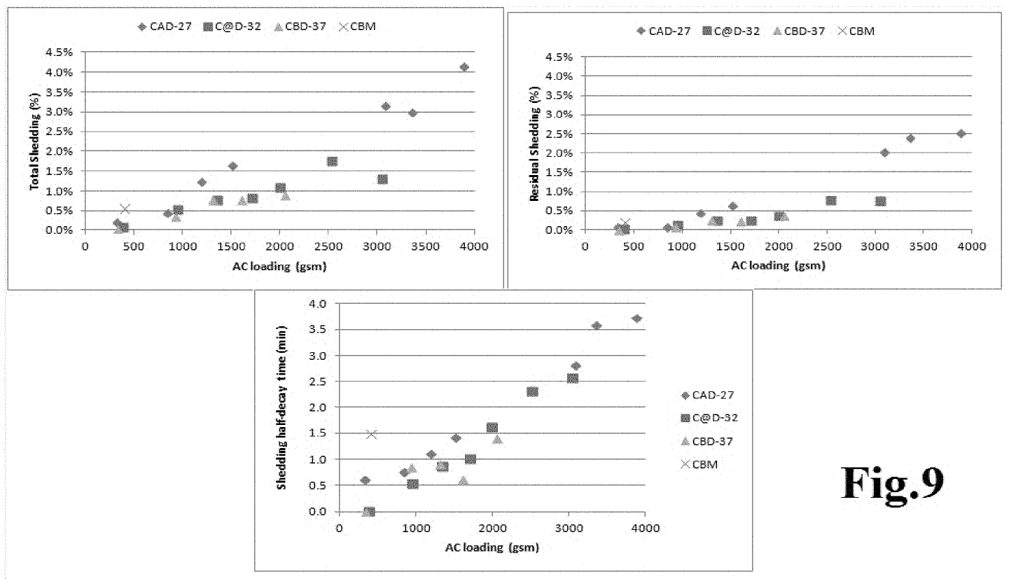

The disclosed structure showed competitive non-shedding properties at high loadings of activated carbon particles. In one series of web structures, activated carbon particles of 50-2000 μm were incorporated at mass concentrations of 59% to 87%; the nonwoven webs had basis weights of 35-50 grams in one square meter, and the weight of the hybrid webs ranged from 103 to 328 g m⁻². In these webs, the filaments did not converge before the collection drum; however, particle retention was 100.00%. In CAD-27 series of web structures, activated carbon particles of 50-710 μm were incorporated at mass concentrations of 88%-99%. The basis weight of CAD-27 hybrid webs ranged from 369-3931 g m⁻², where the weight of the fibrous support was 44 grams per square meter, and the particle retention was more than 97.5%. In C@D-32 webs, activated carbon particles of 50-710 μm were incorporated at mass concentrations of 90%-99%. The basis weight of C@D-32 hybrid webs ranged from 427-3086 g m⁻², where the weight of the fibrous support was 43 grams per square meter, and the particle retention was more than 99%. In CBD-37 webs, activated carbon particles of 50-710 μm were incorporated at mass concentrations of 89%-98%. The basis weight of CBD-37 hybrid webs ranged from 376-2089 g m⁻², where the weight of the fibrous support was 43 grams per square meter, and the particle retention was more than 99.6% (see FIG. 9).

Based on the inventors' knowledge of the prior art, hybrid coform structures of 90-99% mass concentration of particles, at hybrid basis weights of around 4 kilogram in one square meter and particle shedding of less than 1% have not been reported. We believe that this was accomplished because of how and where the filament streams and particle stream met each other.

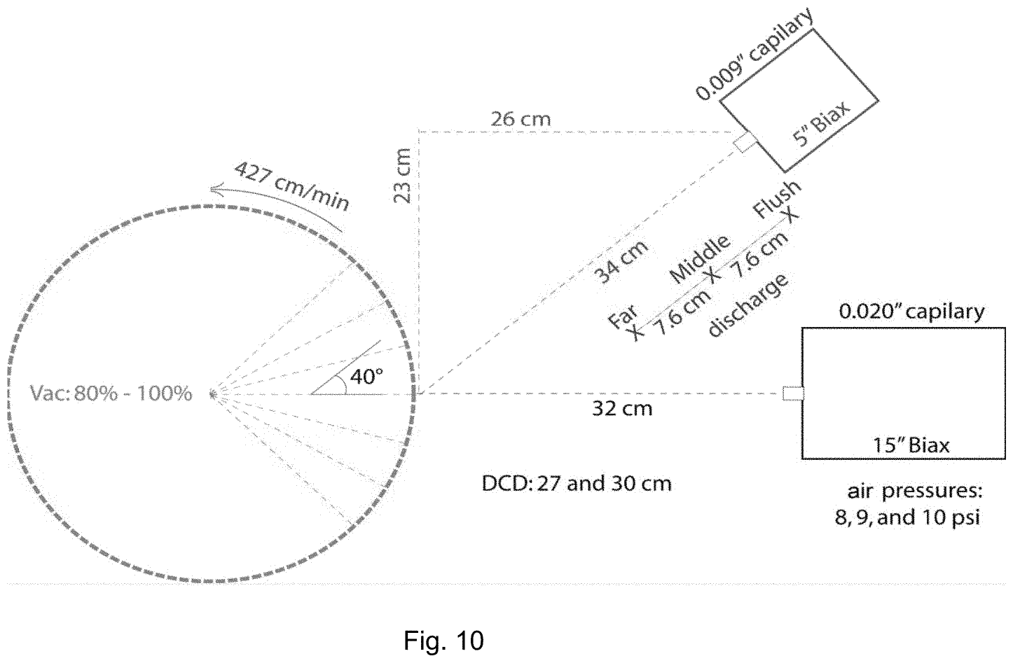

In addition, the location at which the particles were dropped-in the meltblown stream determined their planar distribution, degree of mechanical entrapment, and degree of fusion in the surface of the polymer (see FIG. 10). In one embodiment, particles were introduced to the molten thermoplastic polymer upon the exit of filaments from the die. At this point, filaments were tacky and particles fused in the polymer, which results in the coverage of their surface area. In addition, immediately after the die, the supersonic attenuating airflow creates a turbulent stream and voids of air. Particles tend to fill this void and get disturbed by the turbulence, resulting in less even planar distribution in the web. In another embodiment, particles were dropped-in at the convergence point of the vortices of the two airstreams. These vortices disturbed the planar distribution of the particles, and increased production waste by more than 50%. In the preferred embodiment, particles were dropped-in before the vortices convergence point and after the die, where filaments are semi-solidified. The flow of particles to the filament streams, and their entrainment were not disturbed at this point (zero waste). In addition, more than 95% of the particle attachment to the nonwoven web was through mechanical entrapment. Heat bonding did not happen at this point since filaments were semi-solidified.

Particles were not heated before they hit the web. It is reported in prior art that heating the particles before their introduction to the web, would help joining/bonding them to the filaments. However, heat-bonding the particles to the web results in undesirable loss of active functional surface area. By measuring the specific surface area of the particles before and after incorporation in the web, we confirmed that loss of the surface area was negligible (<5%), and mechanical entrapment was the primary means for particle retention, not heat-bonding.

To our knowledge, the effect of incorporation means on the fiber/web formation has not been reported in the prior art; these are active means such as fluidized particle jet and the same that are accompanied by the discharge device. In several geometries and process parameters, we confirmed that the discharge device (chute) did not influence the webs' basic properties, such as basis weight, basis weight uniformity, fiber diameter, air permeability.

The thermoplastic polymer that was used in this example was polypropylene; however, other suitable thermoplastics include those wherein the thermoplastic polymer is selected from the group consisting of: polyesters, polyamides, thermoplastic copolyetherester elastomers, polyolefines, polyacrylates, thermoplastic liquid crystalline polymers, PBT, Hytrell, and Elastolan.

The sorptive particles of this approach were phosphoric acid impregnated activated carbon; however, other suitable particles include but not limited to superabsorbent fibers and particles, absorbent particles and fibers, synthetic polymer particles, treated activated carbon particles, untreated activated carbon particles, carbon black pigments, metal, detergent, surfactant, biocide, foam, microcapsules, anion exchange resin, cation exchange resin, molecular sieve, modified starches and acrylic polymers, crimped bulking staple fibers, electrically conductive fibers (particle), colorants, herbicides, spores fungicides, rodenticides, odor repellents for animals, bacteria, silicas, aluminas, titanias, zeolites, sodium carbonates, sodium bicarbonates, sodium phosphates, zinc and copper sulfates, cellulose, wood pulp fibers, pulp, and so forth.

One potential application of the disclosed hybrid coform webs is for toxic air filtration where pressure drop and air permeability of the media are critical. The filtration efficiency and air permeability of the hybrid webs did not drop at particle mass concentrations of less than 3000 g m⁻². This approach is also advantageous with regard to the conservation of active functional surface area of the particles. The specific surface area of the activated carbon particles did not drop significantly (<5%). To our knowledge, these properties have not been reported in the prior art.

The methods disclosed in the present publication are preferably single step methods which means that the production of the nonwoven fabric or web takes place at the same instant and does not require further steps like providing the web with clue or like having thermoplastic material cure before applying a further layer of such material or before feeding the functional components.

It is advantageous if the mixture of the first and second stream of polymeric fibre components and of the tertiary functional components takes place during the meltblow process, which means before or at the instant when the respective material reaches the movable surface which collects and shills the respective material. There are positive effects if the aforementioned material streams converge before reaching the collector surface. There are also positive effects if the functional components are fed in only one of the filament streams. This can be done by aid of gravity. In this case the respective first stream of filaments can be directed in a more or less horizontal way. In most cases, parts of the fed functional components will bounce off the respective filament stream and form waste. One of the reasons for this phenomena is the hot air stream and the respective air pressure which surround the filament stream and which are characteristic for the melt blow process. This means that it is hard to feed hundred percent of this functional material into such a filaments stream. It is advantageous if the functional material is fed into such a filament stream just before the two filament streams converge. In the vicinity of the converging point vortices emerge. These vortices have positive effects on the mixture of the different materials which are going to form the fabric or web.

It is therefore advantageous to vary the parameters of the whole production process so as to enhance the formation of these vortices. This can be done by a variation of the different air pressures applied:

The vacuum air pressure of the collector, and the pressures of the hot air streams which attenuate the filament streams. By adjusting all these parameters positive results with regard to the “non-shedding-properties” of the additional functional components can be achieved. The same applies with regard to the positioning of the different dies with regard to each other and with regard to the collector. The surprising property of the fabric to retain large concentrations of the functional material simply by mechanical means (in general clue is not any more necessary) can be further optimized by the aforementioned measures.

The ability to enwrap particles is increased by the formation of baskets by fibers which enwrap relatively small amounts of functional material. It is of course possible to produce webs of the fabric, which consist of different layers, whereby the layers include different concentrations of functional materials. The performance of the respective webs in the aforementioned shedding tests improves if the outer layers are provided with lower concentrations of the functional material. Therefore, it has advantages to provide the middle layers with the highest concentrations. It is also advantageous to provide the middle layers with the best structure of “baskets” to retain the functional material which is in many cases, a ponderous material which consists of particles of fine particles. The amount of material and enwrapped in a single basket is in most cases very small. A vast majority of these baskets (above 90, 95 or 99%) only contains and therefore retains a very small amount of material which can even be as small as a ppm of the material enwrapped in a square inch of the web. State of the art nonwovens, which are often produced in two-step processes in which two outer layers of sequentially produced nonwoven material enwrap a middle layer of functional material have by far bigger baskets—if one can even speak of baskets in these cases.

### Test Methods

Basis Weight and Loading Measurements

Basis weight measurements were done to examine particle loading, particle distribution uniformity, web uniformity across and along, and degree of potential edge effect(s). The results are an average of ten measurements and expressed in two ways, AC loading gsm (grams of AC particles in one m² of the hybrid nonwoven) and AC loading % (mass ratio of AC particles to hybrid nonwoven). A commercial benchmark (CBM) was used for comparison.

Fiber Diameter

Fiber diameter measurements were obtained from SEM micrographs via ImageJ software. Results are based on 600+ measurements from 10+ images. This test was done to study the impact of DCD and particle loading on fiber diameter distribution (based on the visible fibers on each side of the fabric). The bottom side of the web was labeled as “B” (Biax extruder) and the top side of the web was labeled as “W” (Wayne extruder).

Shedding

Particle stability, structural durability, particle retainability, particle release, particle shake-out, or shedding are some common terminologies in the literature that refer to a similar concept. This important structural characteristic determines how well the particles are attached, intermixed, or comingled with the fibrous component. Typically, the hybrid nonwoven is affixed to a mesh sieve and shaken for a period; the ratio of the mass of released particles to the original sample mass is defined as shedding or shake-out. Important parameters are affixation method, sample size, shaking pattern, and shaking time (5-10 minutes). The shake-out test results of this example are average of seven 5 cm×5 cm samples that were pinned down to a US mesh #10 (2 mm). The Tyler Ro-Tap RX-29 instrument was used to shake the samples until shedding stopped (oscillations per minute: 285, oscillations displacement: 1.1 cm×2.9 cm, taps per minute: 150, hammer weight: 2.5 kg). The amount of shake-out was measured every minute until there was no measurable amount (0.1 mg resolution). Tapping elevated the samples from the mesh sieve, so particles could shed from all six sides of the sample. Zero (or minimal) area coverage and extended shake-out tests ensured vigorous shaking conditions. The results are expressed in two ways, total shedding (shedding throughout the test duration) and residual shedding (total shedding minus shedding in the first minute). It was assumed that the shedding in the first minute was from the cut edges. Shedding half-decay times are presented as well; this is the time it takes for shedding to be reduced by one half. Our extended shake-out test (shake until it stops) enables future comparisons between our results and other results (where shaking is only for a defined period). To our knowledge, shedding half-decay time has never been reported in the prior art.

Filtration Properties

The results represent three measurements on PALAS filtration testing equipment (MFP 3000), with a volumetric flow of 360 lpm through 100 cm² filter area (face velocity: 0.6 m s⁻¹). This test was done to determine if the addition of AC particles to unloaded nonwovens would have adverse impacts on the filtration properties (i.e. increase in pressure drop and/or decrease in filtration efficiency).

Air Permeability

The air permeability of the samples was measured by TextTest FX-3300 instrument. The test area was 20 cm² and results are averages of 10 measurements. This test was done to understand the effect of high loading/packing of AC particles in hybrid nonwovens. The goal was to investigate if particle addition and loading would adversely decrease the media air permeability.

Microporosity and BET Surface Area Analyses

NWI's ASiQ instrument (from Quantachrome, Boynton Beach, Fla.) was used to analyze the surface area and pore characteristics of samples. This test was done to examine the uncovered surface-pore areas. Microporosity analysis data included BET specific surface area (SSABET), total pore volume (Vₜ), volume of mesopores (Vₘₑₛₒ), volume of micropores (V_{mic}), and micropore contribution (V_{mic}/Vₜ). The outgassing protocol and analysis parameters (relative pressure point measurements) were constant for all samples (two replicates per test). The following common outgassing protocol (with ramps of 2° C. min⁻¹) was established and used for all the tests:

60° C. for 30 min→80° C. for 30 min→100° C. for 30 min→110° C. for 6 hours.

One potential application of the disclosed hybrid coform webs is for toxic air filtration where pressure drop and air permeability of the media are critical. The filtration efficiency and air permeability of the hybrid webs did not drop at particle mass concentrations of less than 3000 g m⁻². This approach is also advantageous with regard to the conservation of active functional surface area of the particles. The specific surface area of the activated carbon particles did not drop significantly (<5%). To our knowledge, these properties have not been discussed in the prior art.

### Materials

Polypropylene (melt flow index 30) and ground Ammonasorb activated carbon (AM) from Calgon Carbon Corporation Pittsburgh, Pa. were used for all of the examples. The meltblowing dies were from Biax Fiberfilm Corporation Greenville, Wis. The upper die was 5 inches wide, had 4 rows of capillaries, with a Wayne extruder, and the lower die was 15 inches wide, had 2 rows of capillaries, with a Biax extruder. Tables 1 and 2 present the size range and pore characteristics of the AM particles.

TABLE 1
Ground Ammonasorb size ranges

| Sample | AM-75 | AM-300 | AM-425 | AM-710 | AM-850 |
|---|---|---|---|---|---|
| Size range (μm) | <75 | 300-425 | 425-710 | 710-850 | 850-2000 |

TABLE 2
Pore characteristic of Ammonasorb (AM)

| BET specific surface | Total pore | Mesopore | Micropore | Micropore |
|---|---|---|---|---|
| area (m² g⁻¹) | vol. (cc g⁻¹) | vol. (cc g⁻¹) | vol. (cc g⁻¹) | content |
| 659 | 0.272 | 0.047 | 0.225 | 83% |

### EXAMPLES

Several examples are given below demonstrating the properties of the fabrics produced.

### Example 1. Effect of Incorporation Means, Chute, on Web Properties

Basis weight uniformity

| | Chute | Dies air | | | | |
|---|---|---|---|---|---|---|
| | position | pressure | Mean | CV % | | |
| Sample | [in] | [psi] | [gm⁻²] | Global | MD | CD |
| 1 | NA | 8 | 42 | 4.3% | 7.0% | 1.6% |
| 2 | NA | 9 | 39 | 3.0% | 5.0% | 1.0% |
| 3 | NA | 10 | 40 | 2.5% | 4.0% | 1.0% |
| 4 | 0 | 8 | 40 | 2.3% | 3.9% | 0.6% |
| 5 | 0 | 9 | 50 | 2.6% | 4.1% | 1.2% |
| 6 | 0 | 10 | 35 | 4.2% | 5.7% | 2.6% |
| 7 | 3 | 8 | 41 | 1.4% | 2.2% | 0.6% |
| 8 | 3 | 9 | 43 | 2.6% | 4.0% | 1.3% |
| 9 | 3 | 10 | 39 | 1.6% | 2.3% | 0.8% |
| 10 | 6 | 8 | 42 | 1.4% | 1.9% | 0.8% |
| 11 | 6 | 9 | 42 | 1.4% | 2.4% | 0.3% |
| 12 | 6 | 10 | 47 | 2.3% | 3.3% | 1.3% |

For all of the samples, chute position is according to the dies, vacuum pressure was 80%, and DCD was 30 cm.

Note that positioning the chute in the above positions did not influence the basis weight uniformity (CV %). This confirmation was required since we previously observed drastic uniformity changes when an external object was in the meltblowing vicinity. To our knowledge, this has never been investigated/verified in the prior art.

### Example 2. AC Mass Concentration and Uniformity, Initial and Residual Shedding, and Shedding Half-Decay Time

Particle loading and shedding

| | | | | | | | | Shedding |
|---|---|---|---|---|---|---|---|---|
| | | | | | | | | half- |
| | | | | | | Total | Residual | decay |
| | DCD | Web | AC | | | shedding | shedding | time |
| Sample | [cm] | [gm⁻²] | [gm⁻²] | AC % | CV % | [%] | [%] | [min] |
| 1 | 27 | 44 | 0 | 0.0% | 1.7% | NA | NA | NA |
| 2 | 27 | 369 | 325 | 88.1% | 2.5% | 0.2% | 0.1% | 0.6 |
| 3 | 27 | 889 | 845 | 95.1% | 4.8% | 0.4% | 0.1% | 0.8 |
| 4 | 27 | 1230 | 1187 | 96.4% | 3.7% | 1.2% | 0.4% | 1.1 |
| 5 | 27 | 1558 | 1514 | 97.2% | 3.7% | 1.6% | 0.6% | 1.4 |
| 6 | 27 | 3132 | 3088 | 98.6% | 3.8% | 3.2% | 2.0% | 2.8 |
| 7 | 27 | 3401 | 3357 | 98.7% | 5.3% | 3.0% | 2.4% | 3.6 |
| 8 | 27 | 3931 | 3887 | 98.9% | 5.3% | 4.1% | 2.5% | 3.7 |
| 9 | 32 | 43 | 0 | 0.0% | 1.8% | NA | NA | NA |
| 10 | 32 | 427 | 385 | 90.0% | 3.4% | 0.1% | 0.0% | 0.0 |
| 11 | 32 | 989 | 947 | 95.7% | 2.5% | 0.5% | 0.1% | 0.5 |
| 12 | 32 | 1391 | 1348 | 96.9% | 7.2% | 0.8% | 0.2% | 0.9 |
| 13 | 32 | 1747 | 1704 | 97.6% | 2.8% | 0.8% | 0.2% | 1.0 |
| 14 | 32 | 2039 | 1996 | 97.9% | 3.1% | 1.1% | 0.4% | 1.6 |
| 15 | 32 | 2568 | 2525 | 98.3% | 4.0% | 1.8% | 0.8% | 2.3 |
| 16 | 32 | 3086 | 3043 | 98.6% | 4.3% | 1.3% | 0.8% | 2.6 |
| 17 | 37 | 43 | 0 | 0.0% | 1.9% | NA | NA | NA |
| 18 | 37 | 376 | 333 | 88.5% | 3.3% | 0.0% | 0.0% | 0.0 |
| 19 | 37 | 966 | 923 | 95.5% | 3.6% | 0.4% | 0.1% | 0.9 |
| 20 | 37 | 1350 | 1307 | 96.8% | 3.2% | 0.8% | 0.2% | 0.9 |
| 21 | 37 | 1646 | 1603 | 97.4% | 3.1% | 0.8% | 0.2% | 0.6 |
| 22 | 37 | 2089 | 2046 | 97.9% | 2.8% | 0.9% | 0.4% | 1.4 |
| CBM | NA | 529 | 409 | 77.3% | 2.0% | 0.5% | 0.2% | 1.5 |

For all of the samples, chute position was 3 inches from the dies, AC size was 50-710 μm, vacuum pressure was 100%, and dies pressure was 8 psi.

Note that the average CV % of the hybrid webs is around 3.8%, representing the level of global web uniformity. On average, residual shedding was about half of the total shedding (i.e. half of the total shedding happened in the first minute). Therefore, most of the shedding was associated to the cut edges, not from within the web. Larger DCDs presented lower shedding parameters, translating to the higher chance for particle co-mingling. Comparing Sample #19 to the CBM for instance, while the AC loading was more than double in the disclosed web, all shedding parameters were lower for Sample #19.

### Example 3. Effect of Hybridization on Filtration Properties

Filtration efficiency and pressure drop

| | Chute | Dies air | | | | Pressure | Filtration |
|---|---|---|---|---|---|---|---|
| | position | pressure | Web | AC | | drop | efficiency |
| Sample | [in] | [psi] | [gm⁻²] | [gm⁻²] | AC % | [Pa] | [%] |
| 1 | NA | 8 | 42 | 0 | 0.0% | 323 | 28.9 |
| 2 | NA | 10 | 40 | 0 | 0.0% | 306 | 28.6 |
| 3 | 0 | 8 | 40 | 0 | 0.0% | 332 | 34.1 |
| 4 | 0 | 10 | 35 | 0 | 0.0% | 292 | 33.7 |
| 5 | 3 | 8 | 41 | 0 | 0.0% | 331 | 31.1 |
| 6 | 3 | 10 | 39 | 0 | 0.0% | 312 | 27.8 |
| 7 | 6 | 8 | 42 | 0 | 0.0% | 347 | 35.5 |
| 8 | 6 | 10 | 47 | 0 | 0.0% | 344 | 31.4 |
| 9 | 0 | 8 | 264 | 224 | 85.0% | 254 | 28.8 |
| 10 | 0 | 10 | 239 | 204 | 85.3% | 258 | 29.3 |
| 11 | 3 | 8 | 244 | 203 | 83.4% | 262 | 29.7 |
| 12 | 3 | 10 | 228 | 189 | 83.0% | 295 | 32.7 |
| 13 | 6 | 8 | 144 | 101 | 70.4% | 261 | 28.1 |
| 14 | 6 | 10 | 141 | 94 | 66.5% | 288 | 29.5 |

For all of the samples, chute position is according to the dies, AC size was 50-710 μm, vacuum pressure was 80%, and DCD was 30 cm.

Note that, on average, pressure drop in coform webs is about 16% lower than that of the webs with no AC, which is advantageous for filtration applications from an energy standpoint. Filtration efficiency in hybrid samples decreased by around 5%.

Results represent three measurements on PALAS filtration testing equipment, with a volumetric flow of 360 lpm through 100 cm2 filter area (face velocity: 0.6 m s−1). This test was done to determine if addition of AM particles to unloaded nonwovens would have adverse impacts on the filtration properties (i.e. increase in pressure drop and/or decrease in 152 filtration efficiency). Filtration properties studies were only applicable to September 2015 samples. February 2016 samples were so thick that the instrument could not seal the air duct.

### Example 4. Effect of Hybridization on Air Permeability

Air permeability

| | | | | | | Air |
|---|---|---|---|---|---|---|
| | DCD | Web | AC | | Thickness | permeability |
| Sample | [cm] | [gm⁻²] | [gm⁻²] | AC % | [mm] | [cfm] |
| 1 | 27 | 44 | 0 | 0.0% | 0.59 | 44.3 |
| 2 | 27 | 369 | 325 | 88.1% | 1.80 | 49.0 |
| 3 | 27 | 889 | 845 | 95.1% | 2.73 | 52.4 |
| 4 | 27 | 1230 | 1187 | 96.4% | 3.21 | 49.4 |
| 5 | 27 | 1558 | 1514 | 97.2% | 3.73 | 46.1 |
| 6 | 27 | 3132 | 3088 | 98.6% | 6.10 | 42.2 |
| 7 | 27 | 3401 | 3357 | 98.7% | 6.72 | 37.3 |
| 8 | 27 | 3931 | 3887 | 98.9% | 7.78 | 33.9 |
| 9 | 32 | 43 | 0 | 0.0% | 0.37 | 43.1 |
| 10 | 32 | 427 | 385 | 90.0% | 1.84 | 60.3 |
| 11 | 32 | 989 | 947 | 95.7% | 2.97 | 56.9 |
| 12 | 32 | 1391 | 1348 | 96.9% | 3.48 | 48.3 |
| 13 | 32 | 1747 | 1704 | 97.6% | 3.95 | 51.8 |
| 14 | 32 | 2039 | 1996 | 97.9% | 4.35 | 52.4 |
| 15 | 32 | 2568 | 2525 | 98.3% | 5.27 | 49.2 |
| 16 | 32 | 3086 | 3043 | 98.6% | 6.05 | 44.2 |
| 17 | 37 | 43 | 0 | 0.0% | 0.37 | 46.0 |
| 18 | 37 | 376 | 333 | 88.5% | 1.76 | 55.4 |
| 19 | 37 | 966 | 923 | 95.5% | 2.83 | 55.5 |
| 20 | 37 | 1350 | 1307 | 96.8% | 3.71 | 51.7 |
| 21 | 37 | 1646 | 1603 | 97.4% | 3.85 | 52.4 |
| 22 | 37 | 2089 | 2046 | 97.9% | 4.82 | 48.9 |

For all of the samples, chute position was 3 inches from the dies, AC size was 50-710 μm, vacuum pressure was 100%, and dies pressure was 8 psi.

Note that air permeability of the hybrid samples, which contain less than about 3 kg of carbon per square meter, is higher than their control non-hybrid webs. Same as Example 3, enhanced air permeability is advantageous for filtration applications. This was not expected at such high particle concentrations. It represents the unique structure of the disclosed coform structure, wherein filaments are encompassing individual (or groups of) AC particles, and preventing them from packing and clogging the structure.

The vacuum pressure was 125 Pa, test device.

### Example 5. Effect of Hybridization on Microporosity of AC Particles

Microporosity analysis

| | | Avg. | | | | | | |
|---|---|---|---|---|---|---|---|---|
| | Chute | Pore | | | Composite | AM | Estimated | Potentially |
| | position | Size | MP | P. Vol | SSA | Loading | AM SSA | covered |
| Sample | [in] | (Å) | (%) | (cc g⁻¹) | (m² g⁻¹) | % | (m² g⁻¹) | portion |
| AM- | NA | 8.13 | 97 | 0.35 | 870.5 | NA | NA | NA |
| 425 #1 | | | | | | | | |
| Hybrid 1 | 0 | 8.12 | 97 | 0.28 | 681.5 | 85.0 | 801.9 | 7.9% |
| Hybrid 2 | 3 | 8.12 | 97 | 0.29 | 705.4 | 85.0 | 830.0 | 4.6% |
| Hybrid 3 | 6 | 8.10 | 97 | 0.24 | 597.5 | 72.5 | 824.3 | 5.3% |
| AM- | NA | 8.27 | 96 | 0.27 | 653.0 | NA | NA | NA |
| 425 #2 | | | | | | | | |
| Hybrid 4 | 3 | 8.15 | 97 | 0.24 | 607.9 | 97.9 | 622.2 | 4.7% |

For all of the samples, chute position is according to the dies. MP stands for microporosity, and SSA stands for specific surface area.

Note that hybridization did not cover/mask the surface area of AC particles significantly. Higher covered area in Hybrid 1 case was associated to heat-bonded particles to the tacky filaments, since the particles were introduced right after the die (chute position=0 inches). This is a confirmation of particle co-mingling mechanism, i.e. mechanical entrapment vs. heat-bonding. In addition, it verifies that almost all of the surface area is available for adsorption. Based on the inventors' knowledge, this property was never reported/investigated in the prior art.

1. A high basis weight, especially high particle load, non-shedding hybrid nonwoven fabric comprising:
a first fiber component;
at least a second fiber component;
high mass concentration of sorptive particles;
wherein said fiber components are present in amount of about 10 to 100 grams;
said particles are enwrapped by said fibers and do not migrate in and out of said nonwoven fabric;
said particles are ensnared within the said hybrid nonwoven preferably without the aid of adhesive or adhesive polymer and their surface is fully accessible;
said particles are co-mingled with said fiber(s) to form a multi-layered structure;
said hybrid nonwoven has a generally uniform concentration of said particles through its z-direction.

2. The nonwoven web of claim 1 wherein said functional, e.c. sorptive particles, preferably fine particles, are impregnated activated carbons.

3. The nonwoven web of claim 1 wherein said functional, e.c. sorptive particles, preferably fine particles, are activated carbons.

4. The nonwoven web of claim 1 wherein said functional, e.c. sorptive particles can be of any shape or form.

5. The hybrid nonwoven fabric of claim 1 wherein said particle size can be from 0.1 to 4000 μm.

6. The hybrid nonwoven fabric of claim 1 wherein said fibers have similar or different average fiber diameters ranging from 0.1 μm to 100.

7. (canceled)

8. A self-supporting hybrid nonwoven fabric according to claim 1:
wherein the fiber component(s) enwrap the functional, e.c. sorptive particles, preferably fine particles, to form a co-mingled multi-layered hybrid structure;
said structure is formed in one step and said particles are not sandwiched or laminated between two or several layers;
said fibre structure is preferably uniform across, along, and through.

9. A hybrid nonwoven fabric according to claim 1:
wherein functional, e.c. sorptive particles, preferably fine particles, comprise from at least 40% to 98% by mass of the composite structure;
wherein the nonwoven fabric basis weight is between 40 grams per square meter to 4000 grams or more per square meter;
said particles are not released from the hybrid nonwoven by vigorous shaking, abrasion, friction, absorption, swelling or rapidly flowing passage of gasses or other fluids through the hybrid nonwoven;
said particles' or fine particles' or tertiary functional components' retention is achievable without the aid of binders, adhesives, adhesive polymers, or any post processes such as coating and heat treatment.

10. (canceled)

11. A hybrid nonwoven fabric according to claim 1:
wherein the fiber components retain more than 95.5% of the functional particles when exposed to the residual shedding test.

12. A hybrid nonwoven structure, according to claim 1:
wherein the fabric has a substantially opposed color contrast on each side;
said particles comprise from at least 60% to 99% by mass of the composite structure;
said desired color contrast is achievable without any post processes such as laminating, coating, or spraying.

13. (canceled)

14. The hybrid nonwoven fabric of claim 12 wherein said thermoplastic fibers are from a group of spinnable polymers including polypropylene, polyester, and polylactic acid.

15. The hybrid nonwoven fabric of claim 12 wherein said thermoplastic fibers have similar or different average fiber diameters ranging from 0.1 μm to 50 μm.

16. The hybrid nonwoven fabric of claim 12 wherein said tertiary functional component(s) can be from any shape or form of one or combination of materials including, but not limited to, carbon black, activated carbon, and impregnated activated carbon.

17. The hybrid nonwoven fabric of claim 12 wherein said tertiary functional component(s) size can be from 0.1 to 2000 μm.

18. The hybrid nonwoven fabric of claim 12 wherein said tertiary functional component(s) size can be heated before getting co-mingled with the said fiber components.

19.-25. (canceled)

26. A method for producing high basis weight especially high particle or tertiary functional material load, especially high particle load, but non-shedding, hybrid nonwoven fabric, the method consisting of:
a) producing a stream of first polymeric, preferably thermoplastic fiber component, attenuated by (hot) air or other fluid(s);
b) producing at least a stream of secondary first polymeric, preferably thermoplastic fiber component, attenuated by (hot) air or other fluid(s);
c) introducing a plurality of tertiary functional component(s); and
d) collecting the co-mingled admixture on a moving surface;
Wherein tertiary functional component(s) is preferably continuously or/and passively or actively fed in between the said fiber components as they are melt blown.

27. A method according to claim 26 wherein at least at two of the at least two dies are arranged in a non-axisymmetrical manner with regard to each other.

28. A method according to claim 26 wherein the production of the hybrid nonwoven fabric is a one step procedure with no second step like binding particles with glue or the like required.

29.-39. (canceled)

40. A nonwoven web comprising:
meltblown fibres
a tertiary functional component, which might have a poudrous form (particles or fine particles)
wherein
the fiber components retain more than 95.5%, preferably more than 98% preferably more than 99% or more than 99.5% of the functional, e.g. sorptive particles when exposed to the residual shedding test.

41. The non-woven web of claim 40 wherein more than 85% of the mass of the web are provided by the tertiary functional component, having a poudrous form (particles or fine particles).

42.-47. (canceled)
